(19) United States
(12) Patent Application Publication
(10) Pub. No.: US 2014/0329720 A1
(43) Pub. Date: Nov. 6, 2014

(54) ELECTRONIC-BASED BIOSENSOR

(71) Applicant: Triune IP LLC, Richardson, TX (US)

(72) Inventors: Ross E. TEGGATZ, McKinney, TX (US); David William PROVANCE, Richardson, TX (US)

(73) Assignee: Triune IP LLC, Richardson, TX (US)

(21) Appl. No.: 14/267,678

(22) Filed: May 1, 2014

Related U.S. Application Data
(60) Provisional application No. 61/818,045, filed on May 1, 2013.

(51) Int. Cl. C12Q 1/68

(52) U.S. Cl. CPC C12Q 1/6825; C12Q 1/6804
USPC 506/16; 536/23.1; 530/358; 435/287.2

(57) ABSTRACT

The application describes a biosensor system to measure the presence of single molecules of interest or multiple molecules simultaneously in aqueous solutions without the need for trained personnel or dedicated laboratory space. The system uses a lab-on-a-chip platform to contain and manipulate a biosensor component that, through a series of controlled steps, converts the presence of each of the molecule/s of interest into a unique nucleic acid identifier designated by a specific sequence. The generation of the unique nucleic acid identifier is strictly correlated with the presence of the molecule of interest. The platform contains: multiple, sequential, interconnected chambers; transformer coils that induce magnetic fields; the potential for various electric fields; thermal controls; electronics for measurements; and power control.

### PRIORITY ENTITLEMENT

This application is entitled to priority based on Provisional Patent Application Ser. No. 61/818,045 filed on May 1, 2013, which is incorporated herein for all purposes by this reference. This application and the Provisional Patent Application have at least one common inventor.

### TECHNICAL FIELD

The invention relates to the measurement of molecules of interest from aqueous solutions. More particularly, the invention relates the ability to measure the presence and quantity of multiple medically relevant components from biological fluid through electronics.

### BACKGROUND OF THE INVENTION

It is known that the ability to rapidly determine the presence of certain molecules of interest in biological samples is medically relevant to diagnose disease; diagnose infections by viruses, bacteria and parasites; determine medical histories; determine treatment protocols; track effectiveness of therapies; determine resolution of disease or infection; and determine the overall health of a patient. A disadvantage of many such systems is that the measurement of medically relevant biological material is limited to the measurement of a single molecule of interest for rapid assays. This inherently leads to delays in the acquisition of medically relevant patient information from the need for sophisticated equipment, trained personnel and dedicated laboratory space that requires transport of the biological sample for measuring multiple molecules of interest from the same sample. Molecules of interest can include a wide range of molecules including: metabolites such as glucose; toxins such as venoms from snakes and spiders or poisons (both natural and man-made); drugs such as antibiotics, warfarin and cocaine; and proteins such as antibodies and biomarkers of disease or infections.

Due to these and other problems and potential problems with the current state of the art, improved methods and apparatus, utilizing the disclosed Electronic Based Biosensor would be a useful and advantageous addition to the art. The electronic based biosensor can detect and quantify multiple molecules of interest present in an aqueous solution simultaneously and rapidly with high sensitivity and specificity that functions independently of modern laboratory settings and highly trained individuals. Herein is described a universal platform based on the biosensor component used in a lab-on-a-chip for detecting multiple molecules of interest by converting their presence into unique DNA-based identifiers, which can be readily measured by electronics through changes in the electrical behavior of the interactions with those DNA-based identifiers.

### SUMMARY OF THE INVENTION

In carrying out the principles of the present invention, in accordance with preferred embodiments, the invention provides advances in the arts with novel methods and apparatus directed to providing a biosensor system for the detection and measurement of multiple molecules of interest simultaneously from an aqueous sample without the need for highly trained personnel or dedicated laboratory space through a lab-on-a-chip platform.

According to one aspect of the invention, the aqueous sample can be a biological fluid consisting of blood, blood plasma, saliva, amniotic fluid, cerebrospinal fluid or any other fluid obtained from a living organism.

According to another aspect of the invention, the aqueous sample can be generated from the combination of a dry component with water.

According to one aspect of the invention, in an example of a preferred embodiment, the biosensor system contains a biosensor component that consists of a DNA oligonucleotide chemically synthesized from deoxyribonucleotides in a linear sequence of the four available nucleotides, adenosine (A), cytidine (C), guanosine (G) and thymidine (T).

According to yet another aspect of the invention, preferred embodiments of the system of the invention include a synthetic DNA oligonucleotide that forms a secondary structure from a portion of its sequence commonly referred to as a hairpin structure, which has a segment of double-stranded DNA known in the art as a stem and a segment of single-stranded DNA known in the art as a loop.

According to yet another aspect of the invention, a preferred embodiment of the biosensor system is that the biosensor component consisting of the synthetic oligonucleotide has a segment of bases at the three or five prime end that constitute a unique identifiable sequence.

According to yet another aspect of the invention, an exemplary preferred embodiment of the biosensor system is the combination of the hairpin structure with the unique identifiable sequence.

According to yet another aspect of the invention, preferred embodiments of the biosensor component consisting of a synthetic DNA oligonucleotide that contains within its sequence a portion that forms the stem of the hairpin described immediately above the sequence targeted by both a DNA methyl transferase and a Type IIM DNA restriction enzyme.

According to another aspect of the invention, in an example of a preferred embodiment, the controlled action of a DNA methyl transferase on a target sequence contained within the stem of the hairpin segment described above leads to a modification commonly referred to as methylation such that a methyl group is added to a specific nucleotide within the target sequence.

According to another aspect of the invention, in an example of a preferred embodiment, the action of a Type IIM DNA restriction enzyme is dependent on and sequentially controlled in relation to the activity of the DNA methyl transferase such that the activity permits breakage of the molecular bonds between elements of the biosensor component, which liberates the unique identifiable sequence into a freely mobile molecule with respect to the remaining portion of the biosensor component.

According to another aspect of the invention, in a preferred embodiment, the biosensor component described immediately above also has a modification on the nucleotide on the three or five prime end of the synthetic DNA oligonucleotide that permits the attachment of the biosensor component to a solid support.

According to another aspect of the invention, in an example of a preferred embodiment, the solid support consists of a particle that has paramagnetic characteristics.

According to yet another aspect of the invention, a preferred embodiment of the biosensor system is a lab-on-a-chip platform that receives and mixes the biosensor component and the aqueous solution to be tested in the first chamber. Both positive and negative reactions defined by the presence or absence of the molecule of interest are transferred to the second chamber by means of electrical control. More than one type of positive or negative reaction can occur within first chamber. DNA methyl transferase, present in the second chamber, is mixed through electrical control with the positive and negative reactions transferred from the first chamber. Positive reactions combine with the DNA methyl transferase, while the negative reactions do not. Both positive and negative reactions defined by the presence or absence of the molecule of interest are transferred to the third chamber by means of electrical control. Cofactor/s for DNA methyl transferase and the type IIM restriction enzyme, present in the third chamber, is mixed through electrical control with the positive and negative reactions transferred from the second chamber. Positive reactions allow methylation and cleavage of the biosensor component to release the idDNA identifier/s, while the negative reactions do not. The released idDNA identifier/s, which has an intrinsic electrical charge, are transferred to the fourth well by electrical control, while unreleased idDNA identifiers associated with negative reactions are not. The fourth chamber contains a semiconductor transducer with one or more individual sub-wells that have been manufactured with biomaterial. Unique or similar biomaterial is placed in each of these individual sub-wells. The released idDNA identifiers as a result of positive reactions are mixed through electrical control within the fourth chamber to drive interaction with to any corresponding biomaterial present in the sub-wells of the semiconductor transducer. Thermal control in chamber four allows increases or decreases in temperature to further enhance or diminish interactions between free idDNA identifiers and biomaterial. Correct matches between an idDNA identifier and its corresponding biomaterial will bind through hybridization. Incorrect matches between an idDNA identifier and non-corresponding biomaterial will not bind. As a result, the signal to noise is enhanced to identify all sub-wells that have matching biomaterial bound to idDNA identifiers. Each of these correctly matched sub-wells will have different electrical characteristics that can be differentiated from all unmatched sub-wells. Electrical signal processing can be used on the semiconductor transducers to determine this. The measurements made can indicate the presence, absence or the magnitude of the molecules of interest within the aqueous solution under test. The lab-on-a-chip platform can either be an enclosed system which includes control and signal conditioning, or a lab-on-a-chip platform that is wired or wirelessly connected to the control and signal conditioning.

The invention has advantages including but not limited to one or more of the following; capacity to convert the presence of multiple molecules of interest in an aqueous sample into discrete and unique identifiable sequences simultaneously, the ability to control the steps required to undergo the conversion and the ability to measure the liberated identifiable sequences. These and other advantageous features and benefits of the present invention can be understood by one of ordinary skill in the arts upon careful consideration of the detailed description of representative embodiments of the invention in connection with accompanying drawings.

### BRIEF DESCRIPTION OF THE DRAWINGS

The present invention will be more clearly understood from consideration of the following detailed description and drawings in which:

FIG. 1 is a simplified schematic drawing of an exemplary preferred embodiment of the biosensor component.

FIG. 2 is a simplified schematic drawing showing exemplary preferred embodiments of the biosensor component demonstrating three potential target sequences for DNA methyltransferases and corresponding methylation dependent DNA restriction enzymes.

FIG. 3 is a simplified schematic drawing illustrating another example of a preferred embodiment of the biosensor component attached to a solid state particle in coordination with a biomarker that can interact with a molecule of interest.

FIG. 4 is a simplified schematic drawing of an example of a preferred embodiment of the lab-on-a-chip platform.

FIG. 5 is a simplified schematic three dimensional drawing of an example of a preferred embodiment of the lab-on-a-chip platform.

FIG. 6 is a simplified schematic three dimensional drawing of an example of a preferred embodiment of the electronic temperature control elements according to the invention.

FIG. 7 is a simplified schematic drawing of an example of preferred embodiments of wells and sub-wells consisting of interdigitated electrodes and an array of interdigitated electrodes.

FIG. 8 is a simplified schematic drawing of an example of a preferred embodiment of the biosensor component attached to a solid state particle in coordination with a biomarker representing a molecule of interest that is bound to a molecule present in the aqueous solution under testing in the lab-on-a-chip demonstrating a positive reaction in the first chamber.

FIG. 9 is a simplified schematic drawing of an example of a preferred embodiment of the biosensor component attached to a solid state particle in coordination with a biomarker representing a molecule of interest that is bound to a molecule present in the aqueous solution under testing in the lab-on-a-chip identified by a tertiary molecule coupled to a DNA methyltransferase demonstrating a positive reaction in the second chamber.

FIG. 10 is a simplified schematic drawing of an example of a preferred embodiment of the biosensor component attached to a solid state particle in coordination with a biomarker representing a molecule of interest that is bound to a molecule from a biological sample identified by a tertiary molecule coupled to a DNA methyltransferase and the consequential methylation of the adenosine in the recognition sequence of the DNA methyltransferase, which allows the action of the methylation-dependent DNA restriction enzyme to cleave the biosensor component to release the unique DNA identifier sequence demonstrating a positive reaction in the third chamber.

FIG. 11 is a simplified schematic drawing of an alternative example of a preferred embodiment of the biosensor component attached to a solid state particle in coordination with a capture molecule for a molecule of interest from a biological sample that is recognized by a secondary capture molecule coupled to a DNA methyltransferase and the consequential methylation of the adenosine in the recognition sequence of the DNA methyltransferase, which allows the action of the methylation-dependent DNA restriction enzyme to cleave the biosensor component to release the unique DNA identifier sequence demonstrating a positive reaction in the third chamber.

FIG. 12 is a simplified schematic drawing of side views of a sub-well in chamber four showing an interdigitated electrode coupled with biomaterial that matches a unique idDNA identifier sequence for binding before and after the presence of the idDNA identifier within the sub-well.

FIG. 13 shows a list of potential sequences that can comprise the unique idDNA identifier names and sequences of the biosensor component, with the number of bases (Length) in the sequence and the theoretical temperature of melting (Est. Tm) in degrees Celsius.

References in the detailed description correspond to like references in the various drawings unless otherwise noted. Descriptive and directional terms used in the written description such as right, left, back, top, bottom, upper, side, et cetera, refer to the drawings themselves as laid out on the paper and not to physical limitations of the invention unless specifically noted. The drawings are not to scale, and some features of embodiments shown and discussed are simplified or amplified for illustrating principles and features, as well as anticipated and unanticipated advantages of the invention.

### DESCRIPTION OF PREFERRED EMBODIMENTS

While the making and using of various exemplary embodiments of the invention are discussed herein, it should be appreciated that the present invention provides inventive concepts which can be embodied in a wide variety of specific contexts. It should be understood that the invention may be practiced with various alternative components without altering the principles of the invention. For purposes of clarity, detailed descriptions of functions, components, and systems familiar to those skilled in the applicable arts are not included.

In general, the invention provides for the detection and measurement of single molecules of interest or multiple molecules simultaneously in an aqueous solution and provides power supply, conversion, and control capabilities useful in a variety of applications and systems.

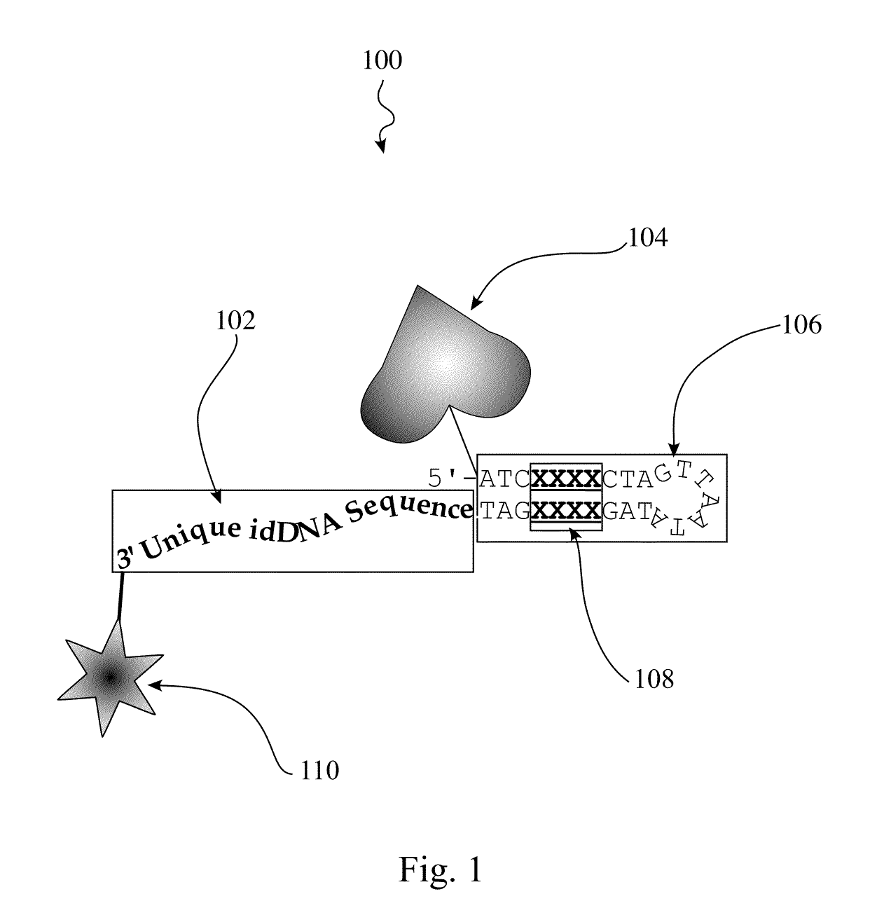

Referring initially to FIG. 1, an example of an exemplary preferred embodiment of the biosensor component is shown. The biosensor component (100) is composed of a DNA oligonucleotide synthesized chemically to present the following characteristics: the absence of methyl groups on all nucleotides comprising the sequence of the oligonucleotide, a segment of nucleotides (102) that together form a unique sequence henceforth referred to as an idDNA identifier, a molecular modification (104) on the nucleotide at the five prime end of the oligonucleotide that permits the attachment of the oligonucleotide covalently or by affinity to another molecule or particle, a sequence that through self-association of the nucleotides forms a hairpin (106) that provides a double-stranded segment, a sequence in the double-stranded portion of the hairpin the constitutes the recognition sequence for binding by both a DNA methyl transferase and a Type IIM restriction enzyme (108) and a molecular modification of the three prime most base that can enhance detection by electronic means by a reduction/oxidation reaction or alternative methods (110). Within the biosensor component, the novel combination of the hairpin (106), the recognition sequence (108) and idDNA identifier (102) creates a means whereby together with the lab-on-the-chip, the biosensor component can attach lo molecules of interest, constituting a positive reaction that results in the controlled release of the idDNA identifier, which is segregated from negative reactions for detection and measurement. Alternatively, the modifications above can be on the nucleotide of the five prime or three prime end of the oligonucleotide. In essence, this biosensor component is an inert molecule that can be attached to a solid support whereby its spatial localization in a system is dependent on said solid support. If the biosensor component interacts with the DNA methyl transferase designated by its target sequence, and the necessary cofactor S-adenosylmethione is present, a specific nucleotide will be modified to include a methyl group. This modification converts the biosensor component to be susceptible to the activity of the Type IIM restriction enzyme, whose activity is to break the molecular bonds in the sequence of nucleotides. By breaking these bonds, the idDNA identifier is no longer attached to the rest of the biosensor component or the solid support. This permits the idDNA identifier to be manipulated independently and allows for its measurement through electronics, which can be enhanced by a modification on a nucleotide within the idDNA identifier sequence.

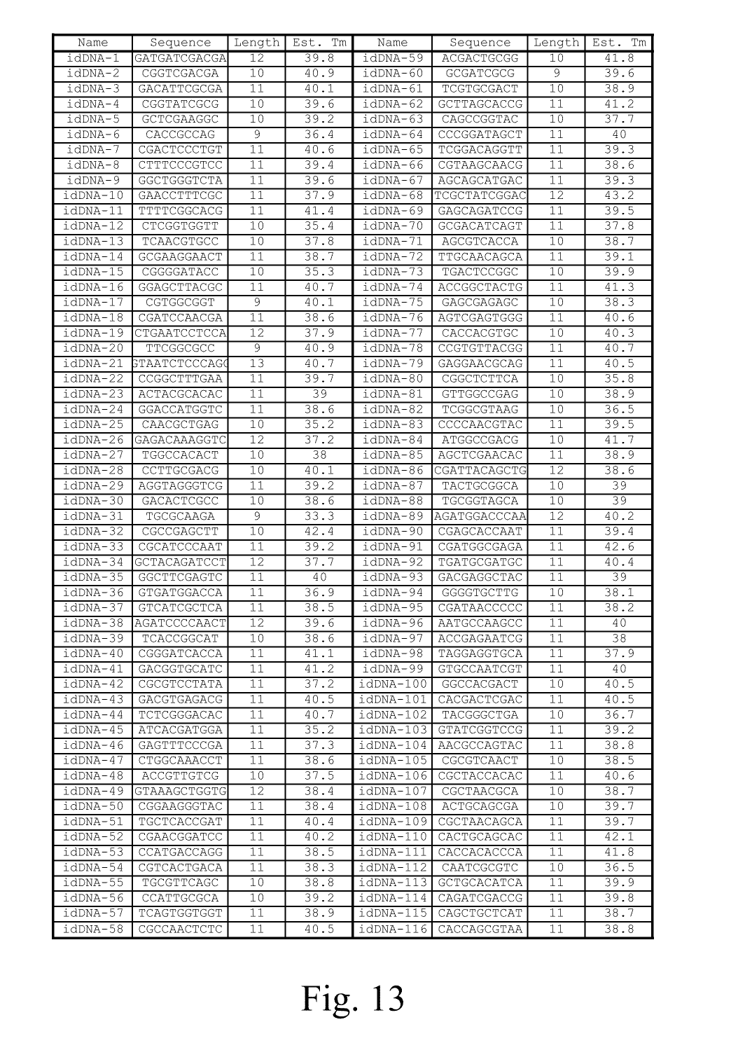

An alternative to the exemplary embodiment, the number and sequence of nucleotides present to form the hairpin can be altered to permit stable self-association in a range of temperatures, preferably ≦55° C. The number and sequence of nucleotides present to form the hairpin can also be altered to permit full activity of both the DNA methyltransferase and the restriction enzyme, which can be as few as three (3) nucleotides on both sides, but can be up to ten (10) nucleotides. The sequence of the idDNA identifier can range between 8 and 30 nucleotides, with a preferred condition such that the melting temperature of the sequence in the idDNA identifier hybridized with its complementary sequence will range around 37° C.±4° C. Each idDNA identifier cannot interact with any other idDNA identifier or their complementary sequence. An example listing of sequences conforming to these conditions is shown in FIG. 13, which is not an exhaustive list.

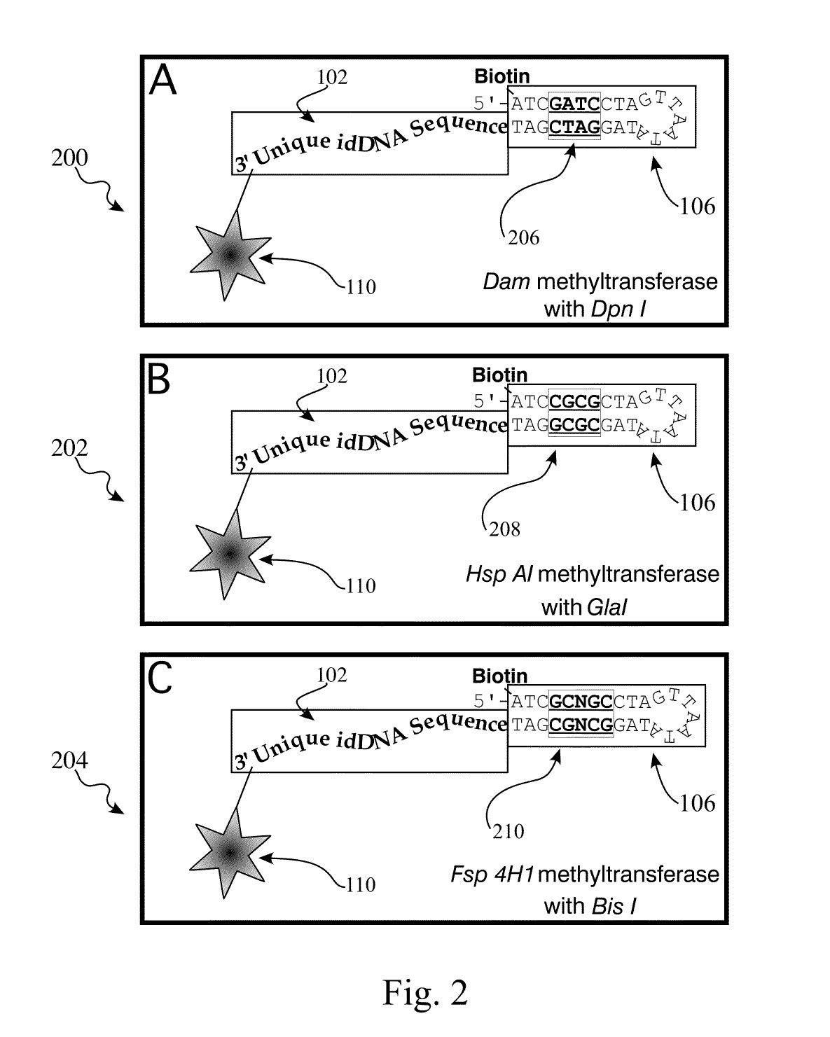

In alternative embodiments of the invention of the biosensor component as depicted in FIG. 2, three alternative sequences are present in a similar fashion as the general biosensor component (108). For each of these three alternative sequences, the sequence is present within the double stranded stem segment of the hairpin (106) in the oligonucleotide. One example can represent the target sequence for Dam methyltransferase (206), which also serves as the recognition sequence of the DpnI restriction enzyme. The presence of the recognition sequence of Dam DNA methyl transferase permits the Dam DNA methyl transferase enzyme, when it is provided with its necessary cofactors, to modify the adenosine in the target sequence with a methyl group. This methylation of the adenosine in the site renders the site competent for suffering from the activity of the restriction enzyme DpnI, which strictly requires the presence of this methyl group for activity. The activity of the restriction enzyme DpnI causes the cleavage of the molecular bond between the adenosine (A) and thymidine (T), which liberates the sequence three prime of the restriction site. Within the biosensor component, this sequence comprises the idDNA identifier.

Additional examples are the sequences in the biosensor component that could also represent the target sequence for Hsp AI methyltransferase and GlaI restriction enzyme (208) or Fsp 4H1 methyltransferase and BisI (210), where “N” represents any nucleotide. Like the Dam DNA methyl transferase, both Hsp AI and Fsp 4H1 require cofactors for methylation activity. In addition, both GlaI and BisI depend on the methylation of the DNA within the target site for activity. In essence, the cleavage activity of the restriction enzymes DpnI, GlaI and BisI requires two, previously occurring events: 1) Presentation of the corresponding DNA methyl transferase and 2) the cofactors required by the respective DNA methyl transferase. Overall, the biosensor system utilizes and leverages these two requirements to effectively distinguish between the presence and absence of molecules of interest within the aqueous sample being tested in the lab-on-a-chip. A collection of biosensor components (100) can be chemically synthesized with the above characteristics that differ in the sequence of the idDNA identifier. FIG. 13 shows an example of 116 specific sequences of the idDNA identifier.

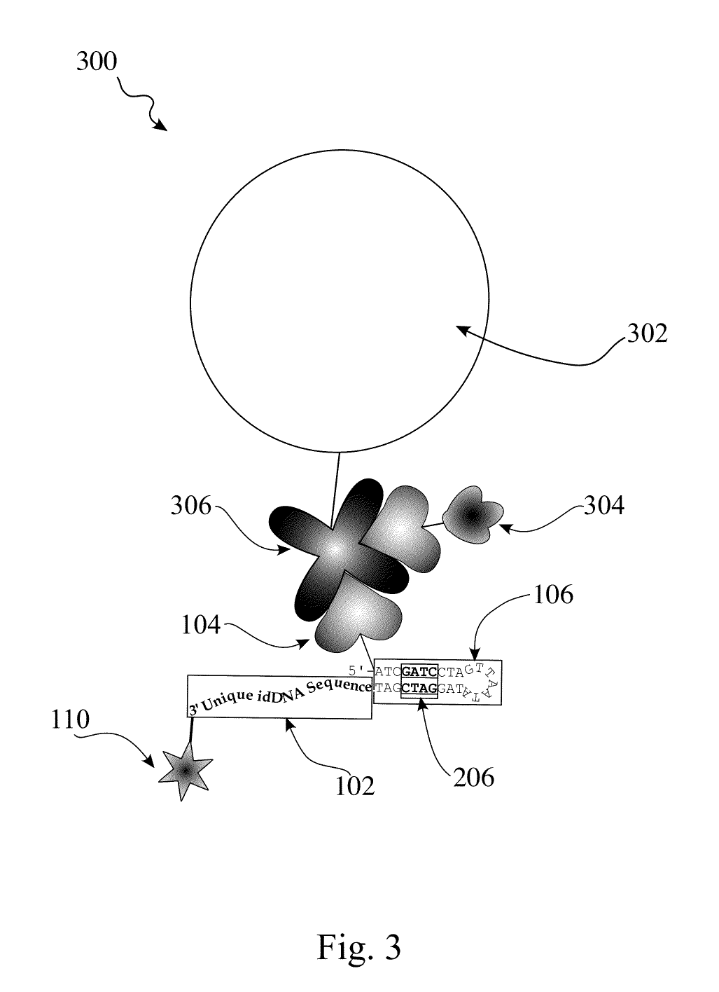

In a preferred embodiment of the invention of the biosensor component as shown in FIG. 3, the oligonucleotide is coupled to a solid state particle (302) in conjunction with biomarker (304) that interacts with the molecule of interest. The solid state particle (302) may be, but is not limited to, a ferrous, semiconductor, metallic, latex, agarose, or saccharide materials. By coupling the biosensor component (100) and the biomarker (304) jointly to the solid state particle, the unique idDNA identifier (102), as defined by its actual sequence, is representative of the molecule of interest once the unique idDNA identifier is liberated from the biosensor component through the sequential and combined actions of the DNA methyltransferase and Type IIM restriction enzymes defined by the target sequence (206). Prior to inclusion on the lab-on-a-chip, the collection of biosensor components (100) will be individually matched with a biomarker (304) by combining with the solid state particle (302). As an example as shown in FIG. 13, this provides an opportunity to measure, but is not limited to, between 1 and 116 different molecules of interest simultaneously.

In an embodiment of the invention, the molecule on the most five or three prime nucleotide of the oligonucleotide (104) can be biotin. The presence of a biotin molecule on the biosensor component allows its attachment by affinity to a solid state particle that is coated with streptavidin. The biotin/streptavidin can be replaced by other chemistries that show tight binding or through the formation of chemical covalent bonds.

In an additional embodiment of the invention, the combination of the biosensor component to the solid state particle in conjunction with the biomarker for the molecule of interest can be performed in varying molar ratios of the biosensor component to the biomarker. Each of the variations of the molar ratio can be related to a specific idDNA identifier such that the sequence of the idDNA identifier relates to the identity of the biomarker and the ratio at which the two were combined. This provides a method to increase the dynamic range of the biosensor for the concentration of the biomarker in the aqueous solution.

In a preferred embodiment of the invention, the combination of the biosensor with the biomarkers of interest, on the solid-state particle, would be performed prior to the addition to the Lab-on-a-chip. This would define a collection that designates the performance capability of the biosensor system to measure specific biomarkers in the aqueous solution.

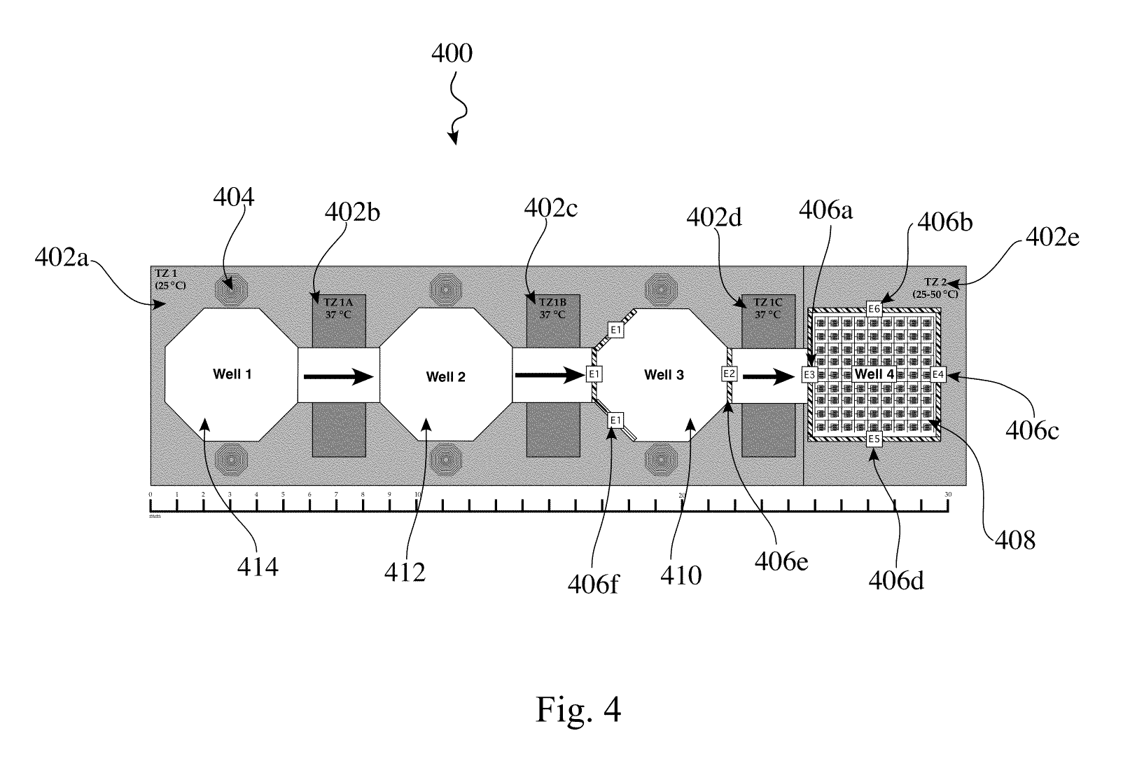

An additional embodiment of the invention includes a Lab-on-a-chip platform (400) constructed of a multiple layer printed circuit board as shown in FIG. 4. The multiple layer printed circuit board consists of electrical and thermally conductive and isolating materials. The design contains transducers (404, 406) capable of inducing electrical or magnetic fields present next to or within the chambers (408, 410, 412, 414) that can be controlled separately to perform mixing and movement between wells of paramagnetic particles (PMPs). The complete Lab-on-a-chip will be actively maintained at a controlled temperature, which is ideally 25° C. using an independently controlled temperature zone (402a) to standardize reaction conditions independent of the ambient temperature. In addition, the channels connecting each chamber (402b, 402c, 402d) can be controlled at lower temperature but can be adjusted and maintained at a higher temperature when transport of the from adjacent cells is required.

Barrier materials are placed in the connecting regions of each chamber (402b, 402c, 402d) which are a solid at lower temperature and become viscous at higher temperatures. Each well is connected to the next by a channel that has a barrier to prevent the transfer of well components that are not specifically and selectively attached to a paramagnetic particle, yet permits transfer of the PMPs. The barrier can be formed by porous material such as, but not limited to, glass, plastic, filter or gel forming materials such as crosslinked polyacrylimide or agarose. In one example, the temperature can be maintained at 37° C. which permits the formation of a gel from thermoreversible substances such as copolymers of ethylene oxide and propylene oxide (tradename Kolliphor® P407).

The area encompassing the last chamber (410) can have temperature control that permits step increases in temperature up to 50° C. (402e). Electrodes (406a, 406b, 406c, 406d, 406e & 406f) are incorporated in various regions of the Lab-on-a-chip platform that can be used cooperatively to generate electric fields of a single polarity or of an alternating polarity. These fields provide attraction and repulsion of liberated idDNA identifiers for mixture and transfer from the third well to the fourth well.

The final well (408) is a semiconductor material which consists of array of many sub-wells as well as many transducers that can be spatially independent for measurement. The transducers may be, but are not limited to, one or more of electro-magnetic, electro-resistive, or electro-static. Each sub-well has bio-material that additionally combines with the idDNA identifiers specifically. The transducers in each sub-well can electrically recognize the unique combination of the bio-material and the idDNA identifiers. This electrical signal is then conditioned by electrical processing and determines the molecule of interest, that could represent a blood borne pathogen or similarly relevant molecule of interest as recognized in the aqueous sample dependent on the location of the transducer within the array.

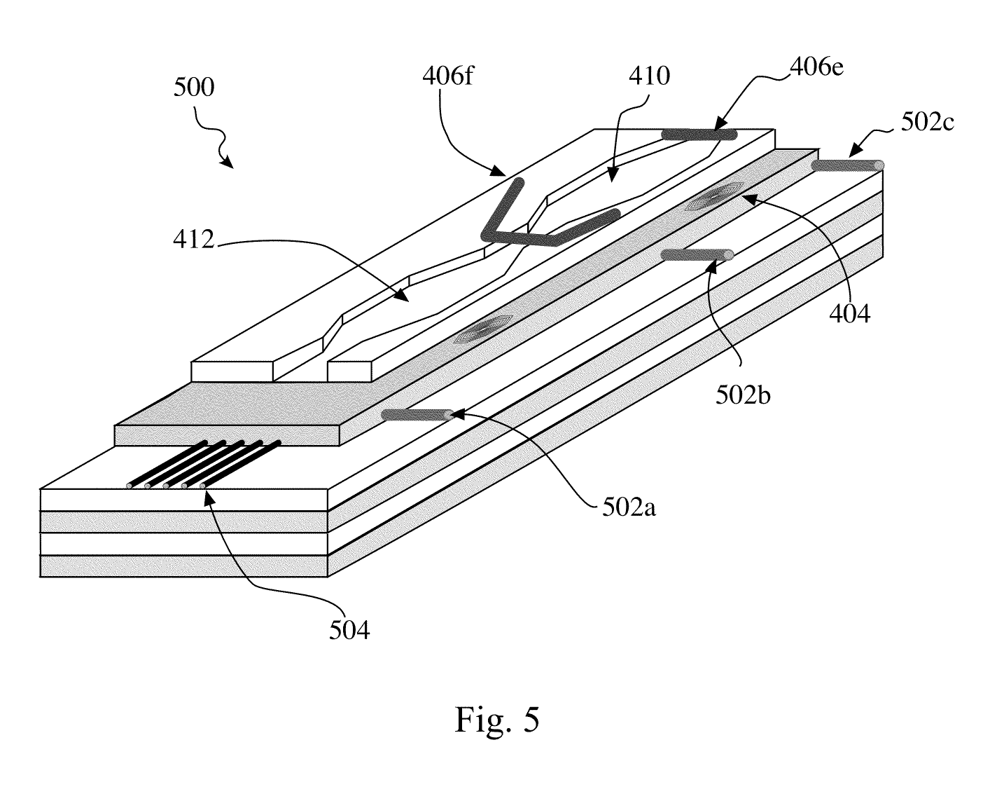

In an alternative representation of the lab-on-a-chip platform shown in FIG. 5, a side-view cut away (500) shows aspects of the layered construction to generate chambers (410, 412), electrodes (406e, 406f), coiled transducers (404), thermal sensors (502a, 502b & 502c) and thermal pipes (504). The placement of the thermal sensors permits a feedback control of the thermal pipes to provide zonal temperature control. All other aspects of the Lab-on-a-chip are consistent with use of the conductive and isolative properties of the multi-layer material make-up used to construct an integrated circuit semiconductor apparatus.

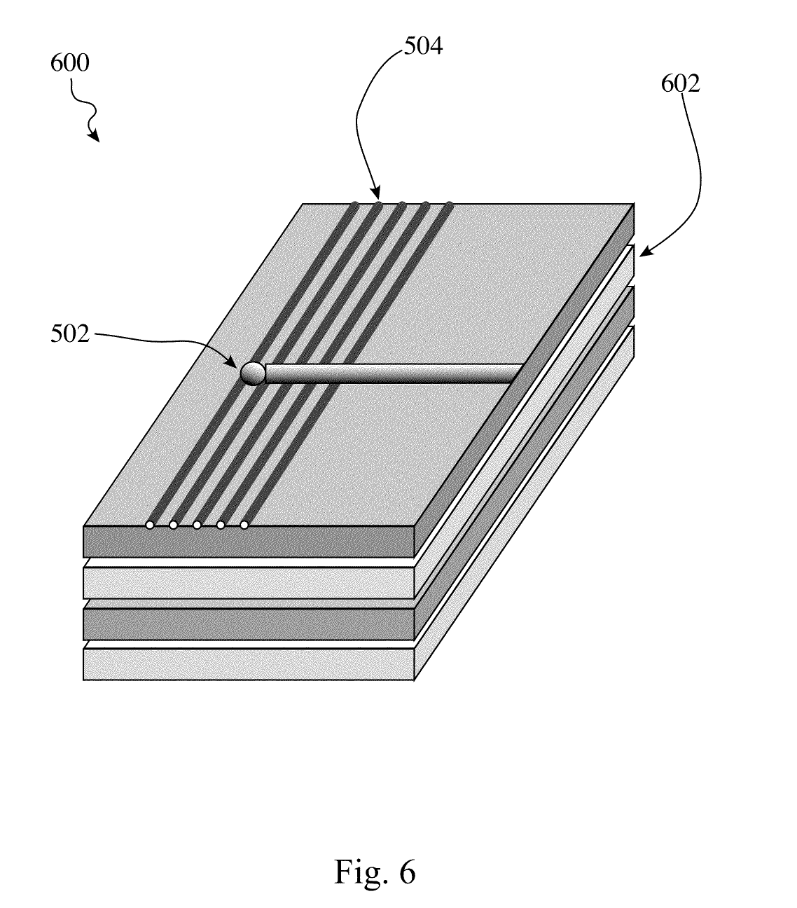

In an additional embodiment of the invention shown in FIG. 6, the placement of the thermal pipes (504) and thermal sensor (502) throughout the construction of the Lab-on-a-chip (LOC) through multiple layers of PC board (602).

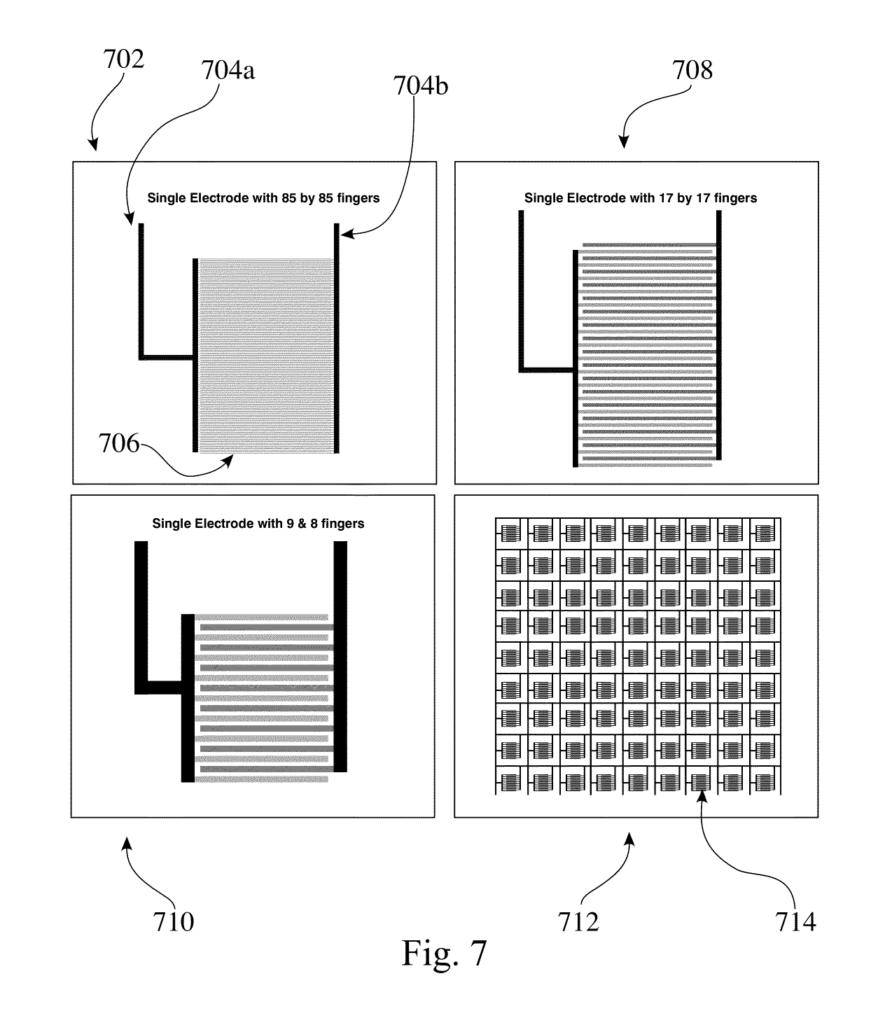

In an alternative embodiment of the invention shown in FIG. 7, the electrodes that measure the presence and quantity of idDNA identifiers are interdigitated electrodes. The general geometry of an interdigitated electrode consists of two principle electrodes (704a & 704b) that exist in parallel to each other that have perpendicular extensions (706) which are referred to as fingers extending in the direction of, but not reaching, the other electrode. The fingers are uniformly sized and spaced from each other. An example of such an interdigitated electrode is (702) that has 85 fingers extending from each parallel electrode. Another embodiment of an interdigitated electrode (708), reduces the number of fingers to 17 extending from each parallel electrode while increasing the width or spacing of each finger. A further embodiment of an interdigitated electrode (710), increases the width or spacing and reduces the number of fingers to 9 from one parallel electrode and 8 from the other electrode. Additionally, other combinations and configuration of the transducers can be adjusted according to the sensitivity of the biomaterials being evaluated. Similarly, other passive, active or micro-electrical machined (MEM) components, such as but not limited to capacitors, inductors, FIN-MOS, J-FETs, CMOS, Bipolar Junction Transistors (BJTs) or cantilevers can be used. Measurements that can be made, but are not limited to, are impedance, pH, electro-magnetic coupling or other types of transducer measurements. This semiconductor material (714) can have the evaluation and control electronics or alternatively the connectors can be brought out to the printed circuit board edge and the evaluation and control electronics can then be connected to the LOC board. This provides the option of a cost saving by not including the more complex evaluation and control electronics within the disposable LOC.

In an embodiment of the invention, the biosensor component consisting of the oligonucleotide (100) can also have present a Reduction/Oxidation moiety, such as methylene blue or ferrocine on the three or five prime most nucleotide that would be maintained with the idDNA identifier/s. These molecules can alter the current registered at electrodes by changes in Faradaic current.

In a preferred embodiment of the invention, the working principle of the biosensor relies on the known fact that cleavage by DpnI and the subsequent release of the unique identifier sequence requires methylation of the DNA at the target site by Dam DNA methyltransferase. As an example, for detection of an antibody against an infectious agent in an aqueous solution, by restricting the activity of Dam to the binding of that antibody to its target within the first chamber of the biosensor lab-on-a-chip platform, liberation of unique identifier sequence can be directly correlated to the presence of that molecule of interest. The biosensor system has been designed to rigidly control methylation by dividing the reaction into a minimum of three separate, sequentially dependent events.

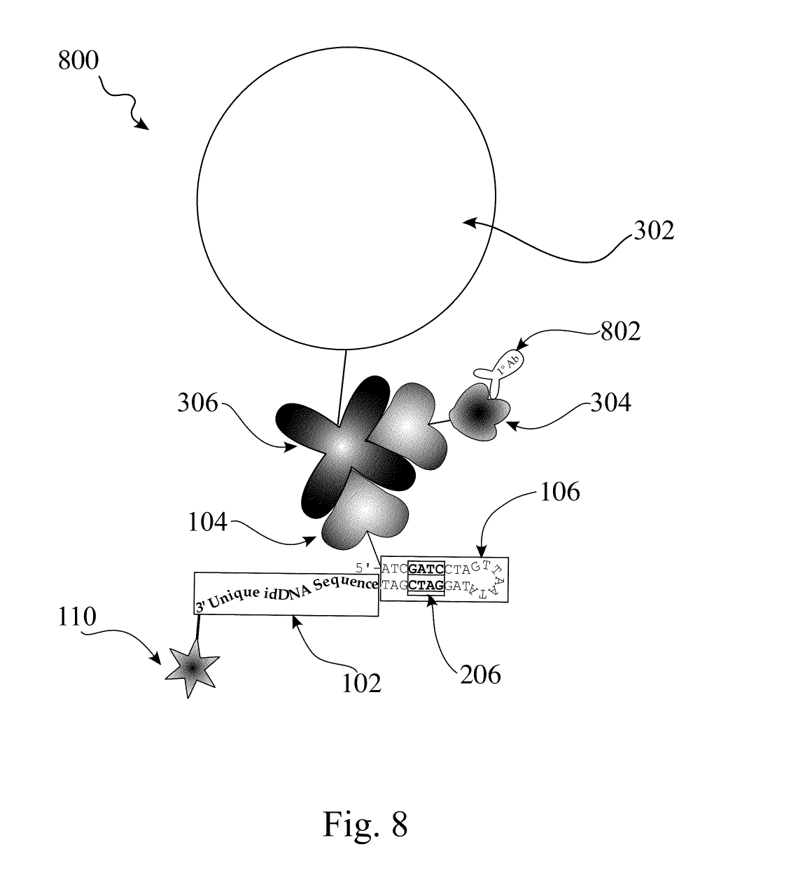

In FIG. 8 is an example of a positive reaction in the first chamber of the LOC, a paramagnetic particle (302) with a surface coupled with streptavidin (306), which can bind to biotinylated biomarker (304) and a biotinylated biosensor component (104) containing a stem and hairpin (106) structure along with the recognition sequence of Dam DNA methyltransferase/DpnI restriction enzyme (206). The sequence of the unique idDNA sequence (102) is matched to the biomarker. When mixed with a biological sample containing an antibody (802) against the biomarker, the antibody binds to the biomarker and becomes incorporated into the complex. Activation of the coil transducers (404) proximal to the first chamber can be used to shift the position of the paramagnetic particle to accelerate binding of potentially present antibody against the biomarker.

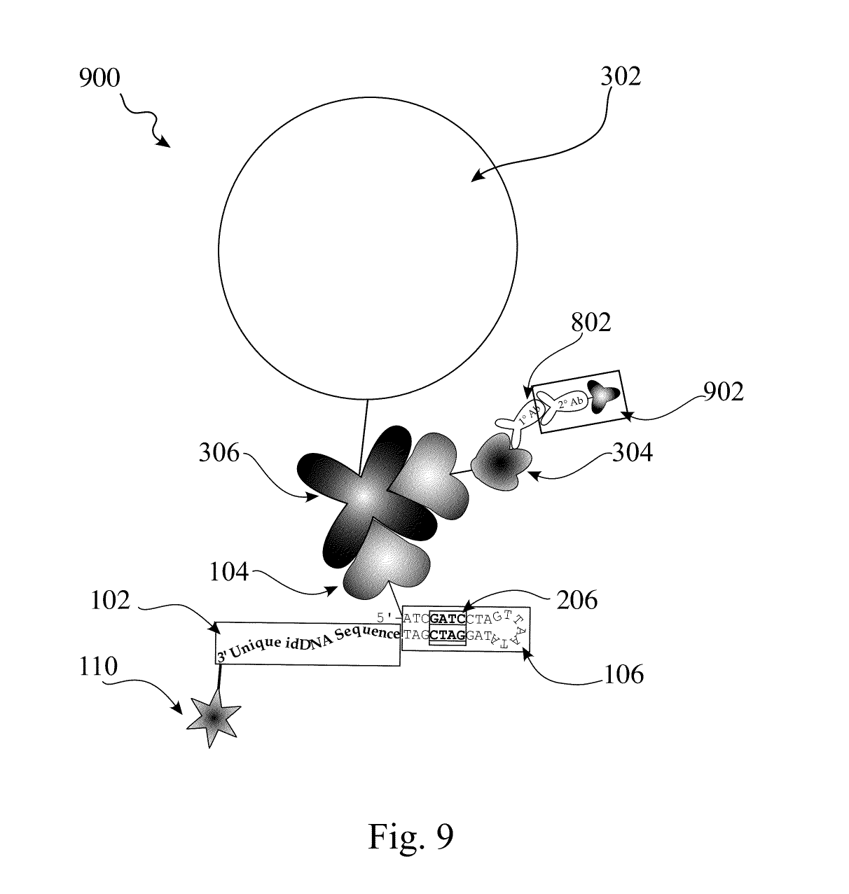

Following a fixed period of time, the coil transducers can be used to transfer the paramagnetic particles and all bound material to the next chamber (412), which contains secondary antibodies that recognize and bind antibodies present in the biological sample shown in FIG. 9. In this example of a positive reaction in the second chamber, these secondary antibodies are coupled to Dam methyltransferase (902), which are inactive.

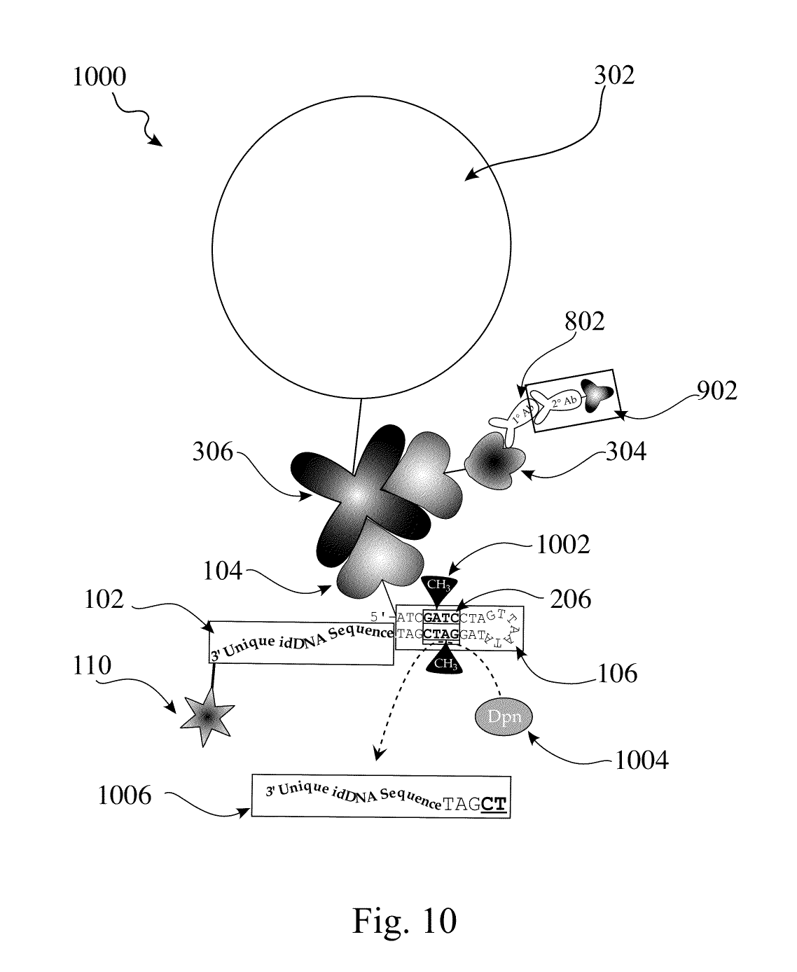

Again, coil transducers provide mixing and transfer of the paramagnetic particles to the third chamber (410), which contains the necessary cofactor S-adenosylmethione that provides the methyl group transferred by Dam methyltransferase activity to the nucleotide and necessary for the activity of the DpnI restriction enzyme. As shown in FIG. 10 for a positive reaction in the third chamber, paramagnetic particles (302) that carry a complex of the biomarker bound to antibodies from the biological sample, also bound by secondary antibody coupled to Dam DNA methyltransferase will methylate the adenosine in the target site (1002), which allows DpnI (1004) to cleave the DNA leading to a free molecule of the unique idDNA sequence (1006) as a result of a positive reaction. This free molecule of unique idDNA sequence can be transported to the fourth chamber of the biosensor platform by electric fields created by the electrodes (406a, 406c, 406e, 406f).

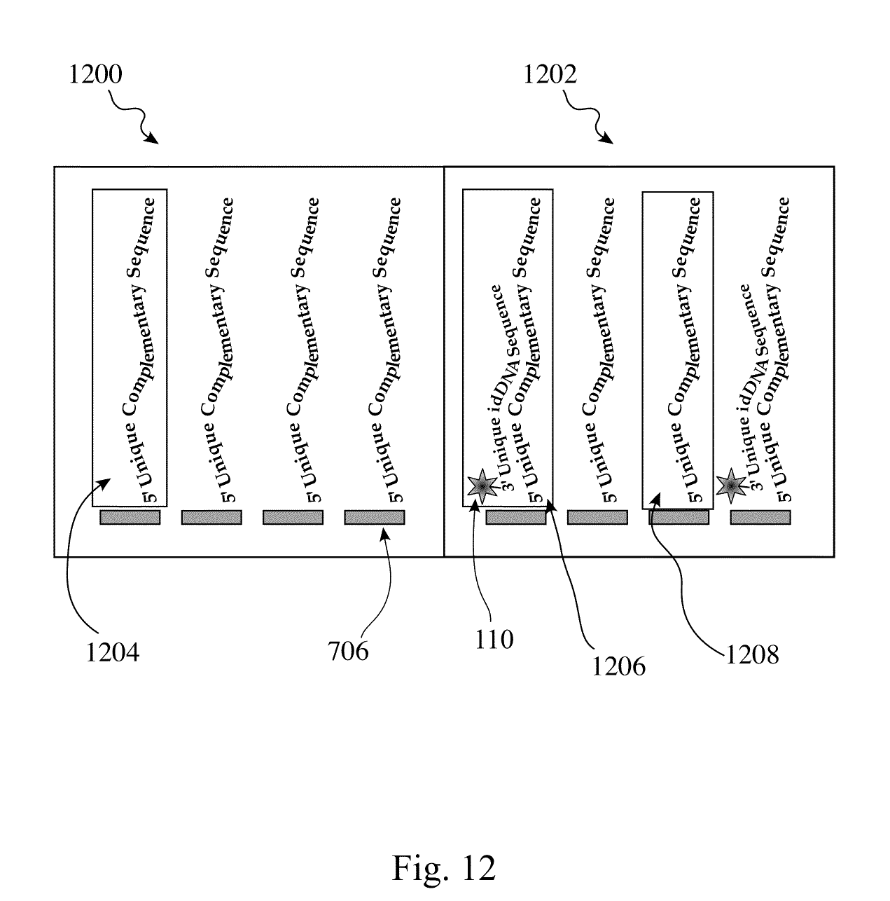

The fourth chamber (408) contains semiconductor material to measure the idDNA identifier. FIG. 12 shows a side view of the surface of the fingers (706) for a given sub-well (1200), which has been manufactured with a complementary sequence of an idDNA identifier (1204). Upon the presence of the idDNA identifier/s as a result of a positive reactions in chamber 3, matching can occur (1206). As a result, electronic measurements can be made to determine the presence or absence of a specific molecule of interest. The magnitude of the measurement can also be determined because the sub-well may not be completely saturated with idDNA identifiers (1208) as a result of positive reactions in chamber 3.

Alternatively, in the final chamber (712) a control sub-well (714) can be measured differentially to an alternative sub-well (714) to determine the presence of idDNA identifiers. Thermal control and electric fields alternatively formed with electrodes (406a, 406b, 406c, 406d) can be used to allow the idDNA sequence to accelerate matching and hybridization to its complementary sequence coupled to a specific position in the interdigitated electrode array (712). The presence of the three prime unique DNA sequence (1202) and hybridization (1206) brings the three prime moiety (110) in proximity to the electrodes and a modulation of the electrical characteristics that are measured. Other unique complementary sequences may not have hybridized to the unique idDNA identifier and the ratio of hybridized and non-hybridized will contribute to the measurement made such that the extent of infection, the presence of infection, the effective treatment and overall health status of a patient is known.

In an alternative embodiment, an additional step can be included whereby a third antibody, which recognizes the secondary antibody, is covalently attached to the surface of chamber three (410) such that paramagnetic particles that experience a positive reaction from chamber one and two (binding of primary antibody and secondary antibody) are retained and the paramagnetic particles that are negative (do not bind primary antibody) are not retained. The unretained, negative paramagnetic particles would collect in a specific region. As such, Dam methyltransferase on the positive paramagnetic particles cannot interact with the biosensor component molecules attached to the surface of negative paramagnetic particles. The inclusion of this alternative embodiment, will avoid false positive measurements.

In an additional embodiment of the inventions, the complementary sequences to the idDNA identifiers are attached directly to the surface of the interdigitated electrode when the interdigitated electrode is constructed of gold by reactive thiol groups.

In order to build the lab on a chip (LOC) device, several transducer and control mechanisms are required. The LOC can be made of printed circuit board material, glass, ceramics, metal, composites, or a combination of these materials. In order to mix or transfer the material within the LOC, several thermal, electro-magnetic, electro-static, and electro-mechanical systems can be used.

For each well configuration, an iso-thermal environment can be achieved through the use of a heating element or a heating/cooling element, such as but not limited to a peltier-cooler. Iso-thermal fields can be made through the use of heat pipes and thermal insulators. Heat pipes can be made of, but not limited to, copper or other metal tracelines through-hole vias or planes. Thermal insulators can be made of the printed circuit board material or other non-thermally conductive materials. In order to control the temperature and to monitor the status of the material in the LOC, sensors are placed locally at areas within the LOC. Thermal sensors, such as thermistors, are placed at critical areas where temperature needs to be monitored such as at the transfer zone (TZ) regions as well as near each of the well regions. Materials can be monitored within each well by evaluating electro-magnetic or electro-static sensors, which can also be used to transfer materials from well to well.

An additional advantage of this design of the biosensor component together with the library of unique identifier sequences and the interdigitated electrode array is to create a universal platform that can be customized by altering the antigens loaded onto the paramagnetic particles such that the device could serve any and all infectious diseases. Antigens can be nearly any molecule that interacts with another. A particular application can be made for antigens that are representative of pathogens and are bound by antibodies in the blood of affected individuals. In this instance, an antigen can be a whole extract of the pathogen, a purified protein/s from the pathogen, a recombinantly expressed protein of the pathogen or a synthetic peptide representing a portion of a protein from the pathogen.

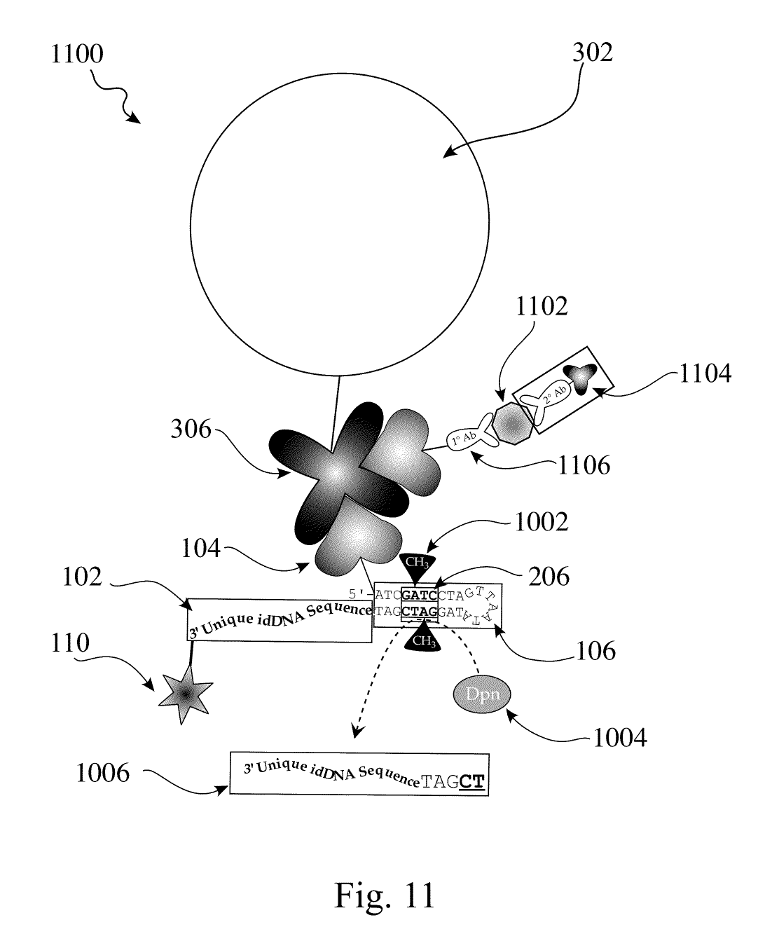

In an additional variation of the invention as shown in FIG. 11, the biotinylated biomarker can be substituted with a biotinylated antibody (1106) against a molecule of interest (1102). The molecule of interest can be a protein, a drug (chemotherapeutic chemical, illicit drugs, etc), a chemical (hormone, pesticide, etc) or the like. Of importance is the availability of a second antibody against the molecule of interest (1104) that can be coupled against the DNA methylase. In a similar manner, the sequential binding of components in a set order leads to the release of the three prime unique DNA sequence.

The invented biosensor system can be expanded to test the presence of multiple molecules of interest simultaneously by the independent formation of the solid support coupled to the biosensor component whose three prime unique DNA sequence and the biomarker or antibody against the molecule of interest such that the sequence of the three prime unique DNA sequence is matched to the molecule of interest. A mixture of three prime unique DNA sequences can be faithfully measured independently in the interdigitated electrode array or similar transducer, where the complementary sequences are knowingly coupled to specific interdigitated electrodes, or similar transducer, within the array.

To further improve performance, mixtures can be made using different target sequences for the DNA methyltransferase/Type IIM restriction enzyme. For instance, the ratio of the quantity of human IgM versus human IgG can provide information concerning the time of onset for an infection. The earliest immune response is preferentially the generation of IgM antibodies. After a period of time after the start of the infection, a person will undergo a seroconversion whereby the immune response changes to the preferential production of IgG antibodies. Using the same biomarker coupled to paramagnetic particles in conjunction with the coupling of the biosensor component having different target sequences in the stem segment of the hairpin whereby one DNA methyltransferase/Type IIM restriction enzyme pair can be directed to detect IgG antibodies and different DNA methyltransferase/Type IIM restriction enzyme pair can be directed to detect IgM antibodies. The readout for the liberation of the three prime unique DNA sequences can indicate whether a person was recently infected or has undergone seroconversion. This information can be used to approximate the length of time of onset for an illness in an individual and thereby determine which persons are most likely contagious. For example, uses for this are applicable to the detection of individuals potentially contagious for an transmittable illness on any transport vehicle allowing for effective remedies to be immediately implemented including, but not limited to: sequestration, isolation, and treatment. Ultimately this type of immediate detection and treatment can prevent tragic pandemics.

Systems solutions for the electronic based biosensor are varied. Due to the fact that the signal conditioning matches a unique idDNA that has an associated antibody to a final target idDNA instead of evaluation of a protein based pathogen, this approach provides an opportunity for greater signal to noise which eliminates the need for an extremely clean lab environment. In addition, biomaterials are used that are commonplace, well established, and are not costly for use. In order to generate a comprehensive result from a test sample, only a few minutes are required and since electrical control systems can be used, limited technical expertise is required to operate such a device. As a result, this product can be used to build systems, including but not limited to, mobile viral test systems, pregnancy tester, blood disease testers, field bio-warfare testers, and similar portable and cost-effective systems.

An additional application of the invention is the use of measurements provided in real time for evaluating biological threats. As an example, registration, tracking and statistical analysis of positive events for specific molecules of interest can be related to the geographical location and/or time of the sample collection, which can provide a means to determine the epidemics of disease or infection and the potential for it to spread throughout a population for the effective implementation of controls.

The methods and apparatus of the invention provide one or more advantages including but not limited to position and types of modifications including on the oligonucleotide, the size and number of chambers in the LOC, types of electrical control, materials make-up of the control system, materials utilized for construction of the LOC, signal conditioning and control. While the invention has been described with reference to certain illustrative embodiments, those described herein are not intended to be construed in a limiting sense. For example, variations or combinations of steps or materials in the embodiments shown and described may be used in particular cases without departure from the invention. Various modifications and combinations of the illustrated embodiments as well as other advantages and embodiments of the invention will be apparent to persons skilled in the arts upon reference to the drawings, description, and claims.

We claim:

1. A biosensor component comprising:
an oligonucleotide comprising:
a nucleotide sequence that forms a hairpin structure;
the nucleotide sequence recognized by a DNA methyl transferase and Type IIM restriction enzyme; and
a segment of nucleotides whose combination forms a uniquely identifiable sequence known as an idDNA identifier.

2. A biosensor component according to claim 1 wherein said nucleotide sequence comprises deoxyribonucleotides.

3. A biosensor component according to claim 1 further comprising a covalently attached biotin modification on a nucleotide.

4. A biosensor component according to claim 1 further comprising a covalently attached moiety that is altered by electrical or magnetic properties.

5. A biosensor component according to claim 3 further comprising a covalently attached moiety that is altered by electrical or magnetic properties.

6. A biosensor component according to claim 1 wherein said nucleotide sequence that forms a hairpin structure is on the five prime end of the oligonucleotide.

7. A biosensor component according to claim 1 wherein said nucleotide sequence that forms a hairpin structure on the three prime end of the oligonucleotide.

8. A biosensor component according to claim 5 further comprising:
a solid state particle;
a biotin molecule connected to a biomarker that represents a molecule of interest; and
a streptavidin.

9. A biosensor component according to claim 5 further comprising:
a solid state particle;
a biotin molecule connected to an antibody that recognizes a molecule of interest; and
a streptavidin.

10. A biosensor component according to claim 8 wherein said solid state particle is a paramagnetic particle.

11. A biosensor system in a Lab-on-a-Chip format formed from electronic compatible material comprising:
a biosensor component;
one or more discrete and interconnected chambers;
a transducer capable of generating electric or magnetic fields;
one or more barriers between interconnected chambers that can be controlled electronically;
one or more temperature zones that can be independently controlled;
one or more sub-wells with biomaterial and transducers capable of generating specific electrical signals.

12. A biosensor system in a Lab-on-a-Chip format according to claim 11 wherein said biosensor components are oligonucleotide comprising:
a nucleotide sequence that forms a hairpin structure;
the nucleotide sequence recognized by a DNA methyl transferase and Type IIM restriction enzyme; and
a segment of nucleotides whose combination forms a uniquely identifiable sequence known as an idDNA identifier.

13. A biosensor system in a Lab-on-a-Chip format according to claim 11 wherein said sub-wells consists of semiconductor material.

14. A biosensor system in a Lab-on-a-Chip format according to claim 11 wherein said electronic compatible material consists of thermally and electrically conductive and isolative properties.

15. A biosensor system in a Lab-on-a-Chip format according to claim 11 wherein said electrical signals are measured and conditioned within the lab-on-a-chip.

16. A biosensor system in a Lab-on-a-Chip format according to claim 11 wherein said barriers consists of a thermoreversible gel.

17. A biosensor system in a Lab-on-a-Chip format according to claim 12 wherein said biosensor component combines with one or more molecules of interest within a chamber.

18. A biosensor system in a Lab-on-a-Chip format according to claim 17 wherein said biosensor component combines with DNA methyl transferase within a chamber.

19. A biosensor system in a Lab-on-a-Chip format according to claim 18 wherein said biosensor component is methylated to a specific nucleotide and is cleaved within a chamber releasing a specific DNA string.

20. A biosensor system in a Lab-on-a-Chip format according to claim 19 wherein said biosensor component is a specific DNA string which matches an associated DNA that is directly correlated to a biomarker that matches to a molecule of interest.


SEQUENCE LISTING (120 sequences)

SEQ ID NO: 1 - DNA, 12 bp, Artificial Sequence
  chemically synthesized oligonucleotide
gatgatcgacga

SEQ ID NO: 2 - DNA, 10 bp, Artificial Sequence
  chemically synthesized oligonucleotide
cggtcgacga

SEQ ID NO: 3 - DNA, 11 bp, Artificial Sequence
  chemically synthesized oligonucleotide
gacattcgcga

SEQ ID NO: 4 - DNA, 10 bp, Artificial Sequence
  chemically synthesized oligonucleotide
cggtatcgcg

SEQ ID NO: 5 - DNA, 10 bp, Artificial Sequence
  chemically synthesized oligonucleotide
gctcgaaggc

SEQ ID NO: 6 - DNA, 9 bp, Artificial Sequence
  chemically synthesized oligonucleotide
caccgccag

SEQ ID NO: 7 - DNA, 11 bp, Artificial Sequence
  chemically synthesized oligonucleotide
cgactccctgt

SEQ ID NO: 8 - DNA, 11 bp, Artificial Sequence
  chemically synthesized oligonucleotide
ctttcccgtcc

SEQ ID NO: 9 - DNA, 11 bp, Artificial Sequence
  chemically synthesized oligonucleotide
ggctgggtcta

SEQ ID NO: 10 - DNA, 11 bp, Artificial Sequence
  chemically synthesized oligonucleotide
gaacctttcgc

SEQ ID NO: 11 - DNA, 11 bp, Artificial Sequence
  chemically synthesized oligonucleotide
ttttcggcacg

SEQ ID NO: 12 - DNA, 10 bp, Artificial Sequence
  chemically synthesized oligonucleotide
ctcggtggtt

SEQ ID NO: 13 - DNA, 10 bp, Artificial Sequence
  chemically synthesized oligonucleotide
tcaacgtgcc

SEQ ID NO: 14 - DNA, 11 bp, Artificial Sequence
  chemically synthesized oligonucleotide
gcgaaggaact

SEQ ID NO: 15 - DNA, 10 bp, Artificial Sequence
  chemically synthesized oligonucleotide
cggggatacc

SEQ ID NO: 16 - DNA, 11 bp, Artificial Sequence
  chemically synthesized oligonucleotide
ggagcttacgc

SEQ ID NO: 17 - DNA, 9 bp, Artificial Sequence
  chemically synthesized oligonucleotide
cgtggcggt

SEQ ID NO: 18 - DNA, 11 bp, Artificial Sequence
  chemically synthesized oligonucleotide
cgatccaacga

SEQ ID NO: 19 - DNA, 12 bp, Artificial Sequence
  chemically synthesized oligonucleotide
ctgaatcctcca

SEQ ID NO: 20 - DNA, 9 bp, Artificial Sequence
  chemically synthesized oligonucleotide
ttcggcgcc

SEQ ID NO: 21 - DNA, 13 bp, Artificial Sequence
  chemically synthesized oligonucleotide
gtaatctcccagg

SEQ ID NO: 22 - DNA, 11 bp, Artificial Sequence
  chemically synthesized oligonucleotide
ccggctttgaa

SEQ ID NO: 23 - DNA, 11 bp, Artificial Sequence
  chemically synthesized oligonucleotide
actacgcacac

SEQ ID NO: 24 - DNA, 11 bp, Artificial Sequence
  chemically synthesized oligonucleotide
ggaccatggtc

SEQ ID NO: 25 - DNA, 10 bp, Artificial Sequence
  chemically synthesized oligonucleotide
caacgctgag

SEQ ID NO: 26 - DNA, 12 bp, Artificial Sequence
  chemically synthesized oligonucleotide
gagacaaaggtc

SEQ ID NO: 27 - DNA, 10 bp, Artificial Sequence
  chemically synthesized oligonucleotide
tggccacact

SEQ ID NO: 28 - DNA, 10 bp, Artificial Sequence
  chemically synthesized oligonucleotide
ccttgcgacg

SEQ ID NO: 29 - DNA, 11 bp, Artificial Sequence
  chemically synthesized oligonucleotide
aggtagggtcg

SEQ ID NO: 30 - DNA, 10 bp, Artificial Sequence
  chemically synthesized oligonucleotide
gacactcgcc

SEQ ID NO: 31 - DNA, 9 bp, Artificial Sequence
  chemically synthesized oligonucleotide
tgcgcaaga

SEQ ID NO: 32 - DNA, 10 bp, Artificial Sequence
  chemically synthesized oligonucleotide
cgccgagctt

SEQ ID NO: 33 - DNA, 11 bp, Artificial Sequence
  chemically synthesized oligonucleotide
cgcatcccaat

SEQ ID NO: 34 - DNA, 12 bp, Artificial Sequence
  chemically synthesized oligonucleotide
gctacagatcct

SEQ ID NO: 35 - DNA, 11 bp, Artificial Sequence
  chemically synthesized oligonucleotide
ggcttcgagtc

SEQ ID NO: 36 - DNA, 11 bp, Artificial Sequence
  chemically synthesized oligonucleotide
gtgatggacca

SEQ ID NO: 37 - DNA, 11 bp, Artificial Sequence
  chemically synthesized oligonucleotide
gtcatcgctca

SEQ ID NO: 38 - DNA, 12 bp, Artificial Sequence
  chemically synthesized oligonucleotide
agatccccaact

SEQ ID NO: 39 - DNA, 10 bp, Artificial Sequence
  chemically synthesized oligonucleotide
tcaccggcat

SEQ ID NO: 40 - DNA, 11 bp, Artificial Sequence
  chemically synthesized oligonucleotide
cgggatcacca

SEQ ID NO: 41 - DNA, 11 bp, Artificial Sequence
  chemically synthesized oligonucleotide
gacggtgcatc

SEQ ID NO: 42 - DNA, 11 bp, Artificial Sequence
  chemically synthesized oligonucleotide
cgcgtcctata

SEQ ID NO: 43 - DNA, 11 bp, Artificial Sequence
  chemically synthesized oligonucleotide
gacgtgagacg

SEQ ID NO: 44 - DNA, 11 bp, Artificial Sequence
  chemically synthesized oligonucleotide
tctcgggacac

SEQ ID NO: 45 - DNA, 11 bp, Artificial Sequence
  chemically synthesized oligonucleotide
atcacgatgga

SEQ ID NO: 46 - DNA, 11 bp, Artificial Sequence
  chemically synthesized oligonucleotide
gagtttcccga

SEQ ID NO: 47 - DNA, 11 bp, Artificial Sequence
  chemically synthesized oligonucleotide
ctggcaaacct

SEQ ID NO: 48 - DNA, 10 bp, Artificial Sequence
  chemically synthesized oligonucleotide
accgttgtcg

SEQ ID NO: 49 - DNA, 12 bp, Artificial Sequence
  chemically synthesized oligonucleotide
gtaaagctggtg

SEQ ID NO: 50 - DNA, 11 bp, Artificial Sequence
  chemically synthesized oligonucleotide
cggaagggtac

SEQ ID NO: 51 - DNA, 11 bp, Artificial Sequence
  chemically synthesized oligonucleotide
tgctcaccgat

SEQ ID NO: 52 - DNA, 11 bp, Artificial Sequence
  chemically synthesized oligonucleotide
cgaacggatcc

SEQ ID NO: 53 - DNA, 11 bp, Artificial Sequence
  chemically synthesized oligonucleotide
ccatgaccagg

SEQ ID NO: 54 - DNA, 11 bp, Artificial Sequence
  chemically synthesized oligonucleotide
cgtcactgaca

SEQ ID NO: 55 - DNA, 10 bp, Artificial Sequence
  chemically synthesized oligonucleotide
tgcgttcagc

SEQ ID NO: 56 - DNA, 10 bp, Artificial Sequence
  chemically synthesized oligonucleotide
ccattgcgca

SEQ ID NO: 57 - DNA, 11 bp, Artificial Sequence
  chemically synthesized oligonucleotide
tcagtggtggt

SEQ ID NO: 58 - DNA, 11 bp, Artificial Sequence
  chemically synthesized oligonucleotide
cgccaactctc

SEQ ID NO: 59 - DNA, 10 bp, Artificial Sequence
  chemically synthesized oligonucleotide
acgactgcgg

SEQ ID NO: 60 - DNA, 9 bp, Artificial Sequence
  chemically synthesized oligonucleotide
gcgatcgcg

SEQ ID NO: 61 - DNA, 10 bp, Artificial Sequence
  chemically synthesized oligonucleotide
tcgtgcgact

SEQ ID NO: 62 - DNA, 11 bp, Artificial Sequence
  chemically synthesized oligonucleotide
gcttagcaccg

SEQ ID NO: 63 - DNA, 10 bp, Artificial Sequence
  chemically synthesized oligonucleotide
cagccggtac

SEQ ID NO: 64 - DNA, 11 bp, Artificial Sequence
  chemically synthesized oligonucleotide
cccggatagct

SEQ ID NO: 65 - DNA, 11 bp, Artificial Sequence
  chemically synthesized oligonucleotide
tcggacaggtt

SEQ ID NO: 66 - DNA, 11 bp, Artificial Sequence
  chemically synthesized oligonucleotide
cgtaagcaacg

SEQ ID NO: 67 - DNA, 11 bp, Artificial Sequence
  chemically synthesized oligonucleotide
agcagcatgac

SEQ ID NO: 68 - DNA, 12 bp, Artificial Sequence
  chemically synthesized oligonucleotide
tcgctatcggac

SEQ ID NO: 69 - DNA, 11 bp, Artificial Sequence
  chemically synthesized oligonucleotide
gagcagatccg

SEQ ID NO: 70 - DNA, 11 bp, Artificial Sequence
  chemically synthesized oligonucleotide
gcgacatcagt

SEQ ID NO: 71 - DNA, 10 bp, Artificial Sequence
  chemically synthesized oligonucleotide
agcgtcacca

SEQ ID NO: 72 - DNA, 11 bp, Artificial Sequence
  chemically synthesized oligonucleotide
ttgcaacagca

SEQ ID NO: 73 - DNA, 10 bp, Artificial Sequence
  chemically synthesized oligonucleotide
tgactccggc

SEQ ID NO: 74 - DNA, 11 bp, Artificial Sequence
  chemically synthesized oligonucleotide
accggctactg

SEQ ID NO: 75 - DNA, 10 bp, Artificial Sequence
  chemically synthesized oligonucleotide
gagcgagagc

SEQ ID NO: 76 - DNA, 11 bp, Artificial Sequence
  chemically synthesized oligonucleotide
agtcgagtggg

SEQ ID NO: 77 - DNA, 10 bp, Artificial Sequence
  chemically synthesized oligonucleotide
caccacgtgc

SEQ ID NO: 78 - DNA, 11 bp, Artificial Sequence
  chemically synthesized oligonucleotide
ccgtgttacgg

SEQ ID NO: 79 - DNA, 11 bp, Artificial Sequence
  chemically synthesized oligonucleotide
gaggaacgcag

SEQ ID NO: 80 - DNA, 10 bp, Artificial Sequence
  chemically synthesized oligonucleotide
cggctcttca

SEQ ID NO: 81 - DNA, 10 bp, Artificial Sequence
  chemically synthesized oligonucleotide
gttggccgag

SEQ ID NO: 82 - DNA, 10 bp, Artificial Sequence
  chemically synthesized oligonucleotide
tcggcgtaag

SEQ ID NO: 83 - DNA, 11 bp, Artificial Sequence
  chemically synthesized oligonucleotide
ccccaacgtac

SEQ ID NO: 84 - DNA, 10 bp, Artificial Sequence
  chemically synthesized oligonucleotide
atggccgacg

SEQ ID NO: 85 - DNA, 11 bp, Artificial Sequence
  chemically synthesized oligonucleotide
agctcgaacac

SEQ ID NO: 86 - DNA, 12 bp, Artificial Sequence
  chemically synthesized oligonucleotide
cgattacagctg

SEQ ID NO: 87 - DNA, 10 bp, Artificial Sequence
  chemically synthesized oligonucleotide
tactgcggca

SEQ ID NO: 88 - DNA, 10 bp, Artificial Sequence
  chemically synthesized oligonucleotide
tgcggtagca

SEQ ID NO: 89 - DNA, 12 bp, Artificial Sequence
  chemically synthesized oligonucleotide
agatggacccaa

SEQ ID NO: 90 - DNA, 11 bp, Artificial Sequence
  chemically synthesized oligonucleotide
cgagcaccaat

SEQ ID NO: 91 - DNA, 11 bp, Artificial Sequence
  chemically synthesized oligonucleotide
cgatggcgaga

SEQ ID NO: 92 - DNA, 11 bp, Artificial Sequence
  chemically synthesized oligonucleotide
tgatgcgatgc

SEQ ID NO: 93 - DNA, 11 bp, Artificial Sequence
  chemically synthesized oligonucleotide
gacgaggctac

SEQ ID NO: 94 - DNA, 10 bp, Artificial Sequence
  chemically synthesized oligonucleotide
ggggtgcttg

SEQ ID NO: 95 - DNA, 11 bp, Artificial Sequence
  chemically synthesized oligonucleotide
cgataaccccc

SEQ ID NO: 96 - DNA, 11 bp, Artificial Sequence
  chemically synthesized oligonucleotide
aatgccaagcc

SEQ ID NO: 97 - DNA, 11 bp, Artificial Sequence
  chemically synthesized oligonucleotide
accgagaatcg

SEQ ID NO: 98 - DNA, 11 bp, Artificial Sequence
  chemically synthesized oligonucleotide
taggaggtgca

SEQ ID NO: 99 - DNA, 11 bp, Artificial Sequence
  chemically synthesized oligonucleotide
gtgccaatcgt

SEQ ID NO: 100 - DNA, 10 bp, Artificial Sequence
  chemically synthesized oligonucleotide
ggccacgact

SEQ ID NO: 101 - DNA, 11 bp, Artificial Sequence
  chemically synthesized oligonucleotide
cacgactcgac

SEQ ID NO: 102 - DNA, 10 bp, Artificial Sequence
  chemically synthesized oligonucleotide
tacgggctga

SEQ ID NO: 103 - DNA, 11 bp, Artificial Sequence
  chemically synthesized oligonucleotide
gtatcggtccg

SEQ ID NO: 104 - DNA, 11 bp, Artificial Sequence
  chemically synthesized oligonucleotide
aacgccagtac

SEQ ID NO: 105 - DNA, 10 bp, Artificial Sequence
  chemically synthesized oligonucleotide
cgcgtcaact

SEQ ID NO: 106 - DNA, 11 bp, Artificial Sequence
  chemically synthesized oligonucleotide
cgctaccacac

SEQ ID NO: 107 - DNA, 10 bp, Artificial Sequence
  chemically synthesized oligonucleotide
cgctaacgca

SEQ ID NO: 108 - DNA, 10 bp, Artificial Sequence
  chemically synthesized oligonucleotide
actgcagcga

SEQ ID NO: 109 - DNA, 11 bp, Artificial Sequence
  chemically synthesized oligonucleotide
cgctaacagca

SEQ ID NO: 110 - DNA, 11 bp, Artificial Sequence
  chemically synthesized oligonucleotide
cactgcagcac

SEQ ID NO: 111 - DNA, 11 bp, Artificial Sequence
  chemically synthesized oligonucleotide
caccacaccca

SEQ ID NO: 112 - DNA, 10 bp, Artificial Sequence
  chemically synthesized oligonucleotide
caatcgcgtc

SEQ ID NO: 113 - DNA, 11 bp, Artificial Sequence
  chemically synthesized oligonucleotide
gctgcacatca

SEQ ID NO: 114 - DNA, 11 bp, Artificial Sequence
  chemically synthesized oligonucleotide
cagatcgaccg

SEQ ID NO: 115 - DNA, 11 bp, Artificial Sequence
  chemically synthesized oligonucleotide
cagctgctcat

SEQ ID NO: 116 - DNA, 11 bp, Artificial Sequence
  chemically synthesized oligonucleotide
caccagcgtaa

SEQ ID NO: 117 - DNA, 27 bp, Artificial Sequence
  chemically synthesized oligonucleotide
tagnnnngatataattgatcnnnncta

SEQ ID NO: 118 - DNA, 27 bp, Artificial Sequence
  chemically synthesized oligonucleotide
tagctaggatataattgatcctagcta

SEQ ID NO: 119 - DNA, 27 bp, Artificial Sequence
  chemically synthesized oligonucleotide
taggcgcgatataattgatcgcgccta

SEQ ID NO: 120 - DNA, 29 bp, Artificial Sequence
  chemically synthesized oligonucleotide
tagcgncggatataattgatccgncgcta